(19) United States
(12) Patent Application Publication
(10) Pub. No.: US 2007/0178139 A1
(43) Pub. Date: Aug. 2, 2007

(54) VAGINALLY ADMINISTRATABLE PROGESTERONE-CONTAINING TABLETS AND METHOD FOR PREPARING SAME

(76) Inventors: Vladimir Ivanov Yankov, Morristown, NJ (US); Azariah Jossifoff, Ramat Gan (IL)

Correspondence Address: DARBY & DARBY P.C., P.O. BOX 770, Church Street Station, New York, NY, 10008-0770, US

(21) Appl. No.: 11/557,036

(22) Filed: Nov. 6, 2006

Related U.S. Application Data
(63) Continuation-in-part of application No. 11/408,614, filed on Apr. 20, 2006.
Application No. 11/408,614 is a continuation of application No. 11/039,062, filed on Jan. 12, 2005.
Application No. 11/039,062 is a continuation of application No. 10/832,742, filed on Apr. 26, 2004.
Application No. 10/832,742 is a continuation of application No. 09/856,417, filed on Aug. 8, 2001.
Application No. 09/856,417 is a continuation of application No. PCT/IL99/00619, filed on Nov. 17, 1999.

(30) Foreign Application Priority Data
Nov. 18, 1998 (IL) 127129

(51) Int. Cl. A61K 9/22

(52) U.S. Cl. 424/430

(57) ABSTRACT

The present invention discloses a method for preparing a tablet for the vaginal administration of progesterone for systemic use. Tablets prepared by this method are also disclosed. Also disclosed are methods for vaginally administering such tablets three times a day to female patients being treated for infertility or other pregnancy-related conditions and disorders in an IVF program. In addition, disclosed are methods of administering a tablet containing 100 mg of natural progesterone at least three times per day to female patients who require stronger luteal support, e.g., older patients and overweight or obese patients, and patients in a donor oocyte program.

This application is a continuation-in-part of U.S. patent application Ser. No. 11/408,614, filed on Apr. 20, 2006, which is a continuation of U.S. patent application Ser. No. 11/039,062, filed on Jan. 12, 2005, which is a continuation of U.S. patent application Ser. No. 10/832,742, filed Apr. 26, 2004, which is a continuation of U.S. patent application Ser. No. 09/856,417 filed Aug. 8, 2001, which was a U.S. national phase application under 35 U.S.C. §371 of International Patent Application No. PCT/IL99/00619 filed in English on Nov. 17, 1999, and claiming priority from Israel Application 127129 filed on Nov. 18, 1998. Each of these prior applications is incorporated herein by reference in its entirety.

### FIELD OF THE INVENTION

The invention relates to the preparation of pharmaceutical compositions containing progesterone, in particular to methods of preparation of compositions for vaginal delivery of progesterone.

### BACKGROUND OF THE INVENTION

Since its discovery in the 1950's, synthetic oral progesterone has been used for a variety of gynecological conditions. However, androgenic activity inherent in the synthetic compound precludes its liberal use in assisted reproductive technology (ART) because of the threat of teratogenic effects.

Furthermore, synthetic progesterone used in hormonal replacement therapy (HRT) may partially reverse the estrogenic benefits on the cardiovascular system and lipoprotein metabolism (Lobo, Am. J. Obstet. Gynecol. 166 (1992), 1997-2004; Fahraeus et al., Eur. J. Clin. Invest. 13 (1983), 447-453; Ottosson et al., Am. J. Obstet. Gynecol. 151 (1985), 746-750; Knopp, Am. J. Obstet. Gynecol. 158 (1988), 1630-1643; Crook et al., 166 (1992) 950-954).

Natural progesterone is devoid of any androgenic activity that might compromise lipoprotein metabolism or induce teratogenicity. Moreover, it probably has a direct beneficial effect on blood vessels (Jiang et al., Eur. J. Pharmacol. 211 (1992), 163-167).

The major difficulty in utilizing natural progesterone is its route of administration. Oral intake is hampered by rapid and extensive intestinal and liver metabolism, leading to poorly sustained serum levels and low bioavailability (Adlercreutz et al., J. Steroid Biochem. 13 (1980), 231-244; Arafat et al., Am. J. Obstet. Gynecol. 159 (1988), 1203-1209; Whitehead et al., Brit. Med. J. 280 (1980), 825-827; Ottosson et al., Br. J. Obstet. Gynecol. 91 (1984), 1111-1119; Padwick et al., Fertil. Steril. 46 (1986), 402-407; Nahoul et al., Maturitas 16 (1993), 185-202; Nillus et al., Am. J. Obstet. Gynecol. 110 (1971), 470-477; Chakmakjian et al., J. Reprod. Med. 32 (1987), 443-448). Intramuscular injection assures reliable absorption, but is painful, can cause local irritation and cold abscesses (Devroey et al., Int. J. Fertil. 34 (1989), 188-193), and may suffer from low patient compliance.

For these reasons, the vaginal route has become an established way in which to deliver natural progesterone. The progesterone is easily administered to the vagina, which has a large potential of absorption, and also avoids liver first-pass metabolism when delivered to the vagina.

Many vaginal formulations have been assayed, mostly as suppositories (Price et al., Fertil. Steril. 39 (1983), 490-493; Norman et al., Fertil. Steril. 56 (1991), 1034-1039; Archer et al., Am. J. Obstet. Gynecol., 173 (1995), 471-478), gelatin capsules (Devroey et al., Int. J. Fertil. 34 (1989), 188-193; Smitz et al., Hum. Reprod. 2 (1992), 309-314; Miles et al., Fertil. Steril. 62 (1994), 485-490), and recently as bio-adhesive gels (Fanchin et al., Obstet. Gynecol. 90 (1997), 396-401; Ross et al., Am. J. Obstet. Gynecol. 177 (1997), 937-941).

Although the suppositories are easily inserted, they melt at body temperature and lead to disturbing vaginal discharge. Oral gelatin capsules containing micronized progesterone have also been used vaginally (Devroey et al., Int. J. Fertil. 34 (1989), 188-193; Smitz et al., Hum. Reprod. 2 (1992), 309-314; Miles et al., Fertil. Steril. 62 (1994), 485-490), but insertion of a small capsule high into the vagina is difficult and large doses of 600 to 800 mg are needed to achieve adequate plasma concentration (Smitz et al., Hum. Reprod. 2 (1992), 309-314; Miles et al., Fertil. Steril. 62 (1994), 485-490; Bourgain et al., Hum. Reprod. 5 (1990), 537-543).

U.S. Pat. Nos. 5,084,277 and 5,116,619, both to Greco et al., disclose a process for the preparation of a progesterone-containing tablet and tablets so prepared. The Greco et al. process involves wet granulation of progesterone into the tablets. As is well-known in the art, wet granulation processes necessitate several steps in the formulation of the resulting tablets. These steps add considerably to the production costs of tablets produced by wet granulation methods, particularly in comparison to comparable “direct compaction” methods, in which the material of interest is tabletted while dry and which involve fewer steps than wet-granulation methods. Greco et al. employs a wet granulation technique because commercially available progesterone has bulk properties which render it unsuitable for direct compaction in the concentrations necessary for use in ART (typically about 50-100 mg progesterone per 1000 mg tablet). Greco gives no suggestion as to how one might be able to tablet progesterone via a direct-compaction method, which is economically more desirable.

The use of a wet granulation method in the preparation of progesterone-containing tablets also precludes incorporation of an effervescent into the tablet. If the tablet is to be vaginally administered, incorporation of an effervescent would be helpful, since the effervescent would aid in the dissolution of the tablet and absorption of the progesterone into the bloodstream.

It has now been found that vaginal administration of a tablet containing 100 mg of natural progesterone at least three times per day results in an improved rate of pregnancy in female patients undergoing progesterone treatment for infertility and other pregnancy-related conditions and disorders.

It has also been found that vaginal administration of a tablet containing 100 mg of natural progesterone at least three times per day results in an improved rate of pregnancy in those female patients who require stronger luteal support, e.g., older patients and overweight or obese patients.

### SUMMARY OF THE INVENTION

The present invention seeks to provide a method for the production of a tablet for the vaginal delivery of progesterone as well as tablets containing progesterone.

There is thus provided, in accordance with a preferred embodiment of the invention, a method for preparing a tablet for the vaginal administration of progesterone for systemic use, comprising the steps of:

slowly mixing water with micronized progesterone, the total amount of water mixed with the micronized progesterone not exceeding the maximum wetting capacity of the micronized progesterone, whereby to obtain wetted micronized progesterone;

drying the wetted micronized progesterone to a humidity content of substantially 0%, whereby to form substantially dry micronized progesterone;

mixing the substantially dry micronized progesterone with other pharmaceutically acceptable excipients or diluents therefor; and

forming a tablet by direct compaction of the substantially dry micronized progesterone which has been mixed with the other pharmaceutically acceptable excipients or diluents therefor.

There is also provided, in accordance with another preferred embodiment of the invention, a method for preparing a tablet for the vaginal administration of progesterone for systemic use, comprising the steps of:

slowly mixing water with micronized progesterone, the total amount of water mixed with the micronized progesterone not exceeding the maximum wetting capacity of the micronized progesterone, whereby to obtain wetted micronized progesterone;

drying the wetted micronized progesterone to a humidity content of substantially 0%, whereby to form substantially dry micronized progesterone;

mixing the substantially dry micronized progesterone with other pharmaceutically acceptable excipients or diluents therefor, including an effervescent; and

forming a tablet by direct compaction of the substantially dry micronized progesterone which has been mixed with the other pharmaceutically acceptable excipients or diluents therefor, including an effervescent.

There is further provided, in accordance with another preferred embodiment of the invention, a method for preparing a tablet for the vaginal administration of progesterone for systemic use, comprising the steps of:

slowly mixing water with micronized progesterone, the total amount of water mixed with the micronized progesterone does not exceed the maximum wetting capacity of the amount of micronized progesterone, whereby to obtain wetted micronized progesterone;

drying the wetted micronized progesterone to a humidity content of substantially 0%, whereby to form substantially dry micronized progesterone;

sieving a first lubricant to obtain a sieved first lubricant;

mixing the substantially dry micronized progesterone with the sieved first lubricant and a material selected from a first filler or a disintegrant to form a first mixture;

mixing a binder which binds dry particles with the first mixture to form a second mixture;

intimately mixing an effervescent and a first quantity of a second filler to form a third mixture;

sieving the third mixture to obtain a sieved third mixture, and then intimately mixing the sieved third mixture and the second mixture to form a fourth mixture;

intimately mixing the fourth mixture with a second quantity of the second filler to form a fifth mixture;

sieving a second lubricant and a material selected from a saponificant or a third lubricant to obtain, respectively, sieved second lubricant and sieved third lubricant;

intimately mixing the sieved second lubricant and the sieved third lubricant with the fifth mixture to form a sixth mixture; and

tabletting the sixth mixture by direction compaction to form a tablet.

In a preferred embodiment of the invention, the amount of water mixed with the micronized progesterone is between about 25 and 28 wt. % of the amount of micronized progesterone.

In another preferred embodiment of the invention, the water is added to the micronized progesterone at rate of between about 6-9 ml per minute, at a mixing speed of between about 25-33.3 rpm.

In still another preferred embodiment of the invention, the first lubricant is sieved through sieves having a pore size of between about 400 and 450 microns, preferably about 425 microns.

In yet another preferred embodiment of the invention, the third mixture is sieved through sieves having a pore size of between about 400 and 450 microns, preferably about 425 microns prior to mixing with the second mixture.

In a further preferred embodiment of the invention, the sieved second lubricant and the sieved third lubricant are passed through sieves having a pore size of between about 100 and 150 microns, preferably 125 microns, prior to mixing with said fifth mixture.

In a still further preferred embodiment of the invention, the wetted micronized progesterone is dried at a temperature of between about 55° C. and about 60° C.

In another preferred embodiment of the invention, all of the mixing steps are carried out at a temperature of between about 15° C. and 30° C.

In one preferred embodiment of the invention, the first lubricant is silicon dioxide (colloidal anhydrous silica).

In another preferred embodiment of the invention, the material selected from a first filler or a disintegrant is a starch exhibiting good flow properties, such as cornstarch 1500 or other starches derived from corn (maize), potatoes or wheat, as are well known in the art.

In a preferred embodiment of the invention, the binder which binds dry particles is polyvinylpyrrolidone (povidone), e.g. Povidone 30.

In another preferred embodiment of the invention, the second filler is derived from a natural source and is more preferably lactose or is composed principally of lactose (e.g. Ludipress®, which as is well known in the art is a commercially available mixture of polyvinylpyrrolidone and lactose).

In a preferred embodiment of the invention, the effervescent is a mixture of a pharmaceutically acceptable carboxylic or dicarboxylic acid, such as adipic acid or tartaric acid, and a pharmaceutically acceptable salt of HCO₃—, such as sodium bicarbonate. Preferably the acid and bicarbonate are present in an amount providing a molar excess of —COOH groups.

In another preferred embodiment of the invention, the first portion and the second portion of the second filler are of generally the same size.

In one preferred embodiment of the invention, the effervescent is prepared prior to the intimate mixing of the first portion of the second filler with the effervescent. In another preferred embodiment of the invention, the effervescent is prepared in situ as part of the intimate mixing of the first portion of the second filler with the effervescent.

In a preferred embodiment of the invention, the intimate mixing of the first portion of the second filler with the effervescent comprises non-intimately mixing the first portion of the second filler with the effervescent and passing the resultant non-intimately mixed mixture through a sieve having an average pore size between about 400 and 450 microns, preferably about 425 microns diameter to obtain the third mixture.

In a preferred embodiment of the invention, the effervescent comprises between about 6 and 10 wt. %, preferably about 8 wt. % of the tablet.

In one preferred embodiment of the invention, the intimate mixing of the second mixture (containing substantially dry micronized progesterone, a sieved first lubricant and either a first filler or a disintegrant (i.e., the first mixture), plus a binder) with the third mixture (an effervescent plus a second filler) to obtain the fourth mixture is accomplished by non-intimately mixing the second mixture with the third mixture to obtain a non-intimately mixed mixture and sifting the non-intimately mixed mixture through a sieve having an average pore size between about 400 and 450 microns, preferably about 425 microns diameter to obtain the fourth mixture.

In a preferred embodiment of the invention, the second lubricant is selected from magnesium stearate, talc, sodium lauryl sulfate, and phosphates known in the art to function as lubricants.

In another preferred embodiment of the invention, the material selected from a saponificant or a third lubricant is sodium lauryl sulfate.

There is also provided in accordance with another preferred embodiment of the invention a tablet prepared by the steps of: slowly mixing water with micronized progesterone, the total amount of water mixed-with the micronized progesterone not exceeding the maximum wetting capacity of the amount of micronized progesterone, whereby to obtain wetted micronized progesterone; drying the wetted micronized progesterone to a humidity content of substantially 0%, whereby to form substantially dry micronized progesterone; mixing the substantially dry micronized progesterone with other pharmaceutically acceptable excipients or diluents therefor; and forming a tablet by direct compaction of the substantially dry micronized progesterone which has been mixed with the other pharmaceutically acceptable excipients or diluents therefor.

There is also provided in accordance with another preferred embodiment of the invention a tablet prepared by the steps of: slowly mixing water with micronized progesterone, the total amount of water mixed with the micronized progesterone not exceeding the maximum wetting capacity of the amount of micronized progesterone, whereby to obtain wetted micronized progesterone; drying the wetted micronized progesterone to a humidity content of substantially 0%, whereby to form substantially dry micronized progesterone; mixing the substantially dry micronized progesterone with other pharmaceutically acceptable excipients or diluents therefor, including an effervescent; and forming a tablet by direct compaction of the substantially dry micronized progesterone which has been mixed with the other pharmaceutically acceptable excipients or diluents therefor, including an effervescent.

There is also provided in accordance with another preferred embodiment of the invention a tablet prepared by the steps of: slowly mixing water with micronized progesterone, the total amount of water mixed with the micronized progesterone not exceeding the maximum wetting capacity of the amount of micronized progesterone, whereby to obtain wetted micronized progesterone; drying the wetted micronized progesterone to a humidity content of substantially 0%, whereby to form substantially dry micronized progesterone; sieving a first lubricant to obtain a sieved first lubricant; mixing the substantially dry micronized progesterone with the sieved first lubricant and a material selected from a first filler or a disintegrant to form a first mixture; mixing a binder which binds dry particles with the first mixture to form a second mixture; intimately mixing an effervescent and a first quantity of a second filler to form a third mixture; sieving the third mixture to obtain a sieved third mixture, and then intimately mixing the sieved third mixture and the second mixture to form a fourth mixture; intimately mixing the fourth mixture with a second quantity of the second filler to form a fifth mixture; sieving a second lubricant and a material selected from a saponificant or a third lubricant to obtain, respectively, sieved second lubricant and sieved third lubricant; intimately mixing the sieved second lubricant and the sieved third lubricant with the fifth mixture to form a sixth mixture; and tabletting the sixth mixture by direction compaction to form a tablet.

There is also provided in accordance with a preferred embodiment of the invention a tablet comprising between about 6 to 20 wt. % progesterone and between about 5 to 12 wt. % effervescent. In a preferred embodiment of the invention, the 20 tablet comprises between about 8 to 12 wt. % progesterone. In a preferred embodiment of the invention, the tablet comprises between about 6 to 8 wt. % effervescent.

There is also provided a method of administering a tablet containing 100 mg of natural progesterone at least three times per day, via the vaginal route. Surprisingly, this results in an improved rate of pregnancy in female patients undergoing progesterone treatment for infertility and other pregnancy-related conditions and disorders.

In addition, administering a tablet containing 100 mg of natural progesterone at least three times per day results in an improved rate of pregnancy in female patients who require stronger luteal support, e.g., older patients and overweight or obese patients, and patients in a donor oocyte program.

### DETAILED DESCRIPTION OF THE PREFERRED EMBODIMENTS

Definitions

As used herein, “QD” refers to once daily administration of a drug or drug formulation or composition.

As used herein, “BID” refers to twice daily administration of a drug or drug formulation or composition.

As used herein, “TID” refers to three times daily administration of a drug or drug formulation or composition.

As used herein, “BMI” refers to body mass index, calculated as weight in kg divided by the square of height in meters.

As used herein, “ITT Population” refers to the “Intent to Treat” Population, which includes all patients who were randomized and had taken at least one dose of study medication.

As used herein, “Efficacy Population” is the ITT population who also had an embryo transfer.

As used herein, “Per-Protocol Population” is the subset of the Efficacy Population who did not have any major protocol violations (generally those pertaining to exclusionary medications for luteal support).

As used herein, “Biochemical Pregnancy” (BP) refers to a positive test for pregnancy according to a serum pregnancy test.

As used herein, “Clinical Pregnancy” (CP) refers to pregnancy confirmed by the presence of a gestational sac on ultrasound.

As used herein, “Ongoing Pregnancy” (OP) refers to pregnancy with heart motion detected by ultrasound.

As used herein, “treatment success” refers to pregnancy achieved.

As used herein, “treatment failure” refers to pregnancy not achieved.

As used herein, “single-center” refers to a study in which all the patients are treated at a single investigation site, as opposed to “multi-center”, which refers to a study with more than one investigational site.

As used herein, “randomized” refers to a patient who is accepted into a research trial and is randomly assigned to one of the treatment groups.

As used herein, “treatment arm” refers to the treatment group to which a patient is assigned.

As used herein, “ovarian reserve” is a measure of biological competency of the ovary to mature and ovulate oocytes.

As used herein, “open-label” refers to a trial in which the treatment arms are discernable even during the study.

As used herein, the “screening” phase of a clinical trial refers to the period of evaluation before the patient is accepted into the trial and randomized.

As used herein, the “single-day” phase of a clinical trial refers to a study phase when a patient receives up to 24 hours of treatment.

As used herein, the “washout” phase of a clinical trial refers to the time between study phases during which the patient receives no treatment and the previous medication is allowed to clear from the body.

As used herein, the “multiple-day” phase of a clinical trial refers to a study phase when a patient receives more than one day of study medication.

As used herein, “trough level” refers to the concentration just before the next dose of medication is administered.

As used herein, “systemic exposure” refers to the concentration in the circulatory system over the course of a day.

As used herein, “adequate secretory phase endometrium” refers to successful conversion of proliferative endometrium to secretory endometrium in order to allow embryo implantation.

As used herein, “luteal phase” refers to the portion of the menstrual cycle from ovulation to menses.

As used herein, “Body Mass Index” (BMI) is a statistical measure of the weight of a person scaled according to height. BMI is calculated by dividing the patient's body weight (in kg) by the square of the height, and is expressed in the unit kg/m², i.e.,

$$BMI = \frac{weight ( kg )}{height ( m ) \times height ( m )}$$

Using Imperial Units,

$$BMI ( expressed as lb \text{/} {in}^{2} ) = \frac{weight ( lb )}{height ( in ) \times height ( in )} \times 703$$

As used herein, “overweight” refers to female patients having a BMI between about 25.0 and about 29.9 kg/m².

As used herein, “obese” refers to female patients having a BMI≧ about 30 kg/m².

As used herein, “morbidly obese” refers to female patients having a BMI≧ about 40 kg/m².

As used herein, “slowly mixing water with micronized progesterone” refers to water added at a rate of 140 +/−20 g/min.

As used herein, “substantially dry progesterone” refers to progesterone granules dried to no more than about 0.5% w/w moisture at 105° C.

As used herein, “a humidity content of substantially 0%” refers to moisture content ranging from 0% up to about 0.5% w/w.

As used herein, “maximum wetting capacity of the amount of micronized progesterone” refers to about 30% w/w water in wetted micronized progesterone.

As used herein, “sieving” refers to the dry blend (progesterone granules with other excipients) being passed through a 40 mesh screen.

One of ordinary skill in the art would understand the meaning of the terms set forth herein, as well as other relevant terms for which no definition is provided herein.

For an excellent reference relating to clinical trials, refer to Bert Spilker, Guide to Clinical Trials (Raven Press 1991).

For an excellent reference relating to infertility, refer to (1) et al., Reproductive Endocrinology, Surgery, and Technology (Eli Y. Adashi, John A. Rock, M. D., and Zev Rosenwaks M. D. , eds., Lippincott-Raven Publishers 1995); (2) Leon Speroff et al., Clinical Gynecologic Endocrinology and Infertility (Wolters Kluwer 1999); and (3) Jerome F. Strauss III et al., Yen and Jaffe's Reproductive Endocrinology—Physiology, Pathophysiology, and Clinical Management (W. B. Saunders 2004).

There is also provided a method of administering a tablet containing 100 mg of natural progesterone at least three times per day. It has been unexpectedly discovered that TID administration of a vaginal progesterone tablet prepared according to this invention results in an improved rate of pregnancy in female patients undergoing progesterone treatment for infertility and other pregnancy-related conditions and disorders. This is surprising because BID administration of a vaginal progesterone tablet prepared according to this invention has been found to be as effective as the current approved therapy, Crinone, an 8% progesterone gel (Serono International S.A., Geneva, Switzerland) administered 90 mg QD. For this reason, further improvement over BID administration of a vaginal progesterone tablet prepared according to this invention was not expected; hence it was surprising to see the improved rate of pregnancy seen upon TID administration of the same vaginal progesterone tablet prepared according to this invention.

In addition, administering a vaginal progesterone tablet prepared according to this invention at least three times per day results in an improved rate of pregnancy in female patients who require stronger luteal support, e.g., older patients and overweight or obese patients. This is significant because this results in improved pregnancy rates for these harder to treat populations.

The vaginal progesterone tablets according to this invention are made by slowly mixing water with micronized progesterone to obtain wetted micronized progesterone, followed by drying the wetted micronized progesterone to form substantially dry micronized progesterone. The substantially dry micronized progesterone is then mixed with an effervescent and one or more other non-effervescent ingredients, including, but not limited to, pharmaceutically acceptable excipients, diluents, lubricants (including saponificants), fillers, binders and disintegrants. Once all the ingredients have been mixed, they are formed into a tablet by direct compaction. The order of addition of the ingredients other than the substantially dry progesterone and the effervescent can vary slightly without compromising the usefulness of resulting tablet. However, it is crucial that the effervescent be added only to substantially dry ingredients, e.g., substantially dry progesterone, and that wet ingredients are not added subsequent to the addition of the effervescent, because the effervescent will effervesce if exposed to wetness. If this occurs, compaction into a tablet will be impossible. Furthermore, it is crucial that the tablet is kept dry until it is administered to a subject being treated, because once wet, the tablet will begin to effervesce and prematurely release the progesterone.

The vaginal progesterone tablets according to this invention are administered directly to the vagina of a subject being treated for infertility or other pregnancy-related conditions and disorders. In other words, the tablet is placed directly in the vagina of the subject being treated. The tablet may be self-administered by the patient or may be administered by someone other than the patient, and may be administered by hand or by using a device designed for direct placement of an effervescent tablet in the vagina.

The invention will be better understood through the following illustrative and non-limitative description and examples of preferred embodiments of the invention.

### EXAMPLE 1

Preparation of Tablets

Step 1. To 1000 g of micronized progesterone were added 280 g of distilled water, with mixing using a planetary mixer, over a period of 30 minutes. After mixing, the wetted micronized progesterone was spread on pans to thickness of about 4-5 mm, and the pans then placed in an oven at 58° C. The humidity was checked periodically using a humidity checker. When the humidity of the micronized progesterone was reduced to substantially 0%, the dried micronized progesterone was either used immediately in step 2 as described below, or was stored in dry, sealed containers for later use in step 2.

Step 2: Colloidal anhydrous silica (Aerosil 380, 25 g) was sieved through a Russel sieve having pores of 425 micron size, and mixed for 10 minutes with 1000 g of micronized progesterone from Step 1 and 2100 g of maize 1500 starch, using an Angelsman mixer at 32 RPM, to form Mixture A. At the end of the 10 minutes of mixing, 490 g of povidone 30 were added to Mixture A, and mixing was continued for another ten minutes, to prepare “Mixture B”.

Step 3: Lactose (Ludipress®, BASF, 3800 g), adipic acid (570 g) and sodium bicarbonate (430 g) were mixed for 10 minutes at room temperature using an Angelsman mixer at 32 RPM. Following mixing, these ingredients were sieved through a Russel sieve having pores of 425 microns to obtain “Mixture C”.

Step 4: Mixtures B and C were mixed for 10 minutes at room temperature using an Angelsman mixer at 32 RPM to obtain “Mixture D”.

Step 5: Mixture D (8415 g) was mixed with 3800 g of lactose (Ludipress®) for 10 minutes at room temperature using an Angelsman mixer at 32 RPM, to obtain “Mixture E”.

Step 6: Magnesium stearate (230 g) and sodium lauryl sulfate (50 g) were sieved through a Russel sieve (pore size 125 microns). The sieved magnesium stearate and sodium lauryl sulfate were then mixed for with mixture E for 20 minutes at room temperature using an Angelsman mixer, to obtain “Mixture F”.

Step 7: Tablets were obtained from mixture F by direct compaction using an Eko Korsch Press. The amounts of ingredients listed in this example are suitable for production of 10,000 tablets each containing about 100 mg progesterone.

### EXAMPLE 2

Using the above process, tablets of 1187 mg to 1312 mg total weight, containing from 90 to 110 mg progesterone, were obtained.

### EXAMPLE 3

The process described in Example 1 was modified by doubling the amount of filler (Ludipress®) to obtain tablets containing on average 50 mg progesterone.

### EXAMPLE 4

The pharmacokinetics and clinical use of tablets prepared in accordance with the invention were evaluated as follows: 50 healthy, post-menopausal women with intact uteri, 39 of whom had suffered premature menopause and 11 who were truly postmenopausal, all of whom were undergoing hormone replacement therapy (HRT), submitted blood samples for determination of baseline profiles of hormones (progesterone and other hormones) and other biochemicals (bilirubin, cholesterol, etc.). The blood samples were taken at 8 AM on the first day of the evaluation (day 0) in a fasting state, by intravenous indwelling catheter which was introduced into the cubital vein. Non-estrogen primed postmenopausal women were chosen in order to avoid confusion with endogenous progesterone secretion and estrogen influence on vaginal mucosa absorption (Villanueva et al., Fertil. Steril. 35 (1981), 433-437).

The women then self-administered the progesterone vaginal tablet using a plastic applicator and lay down for 20 minutes. Repeat blood samples for progesterone concentration were withdrawn 0.5, 1, 2, 4, 6, 8, 10, 12 and 24 hours after the vaginal insertion. Blood was allowed to clot at room temperature for 1 hour, after which the serum was separated by centrifugation and stored at −20° C. until analysis.

To evaluate clinical use of the drug, the women were instructed to insert tablets prepared in accordance with the present invention, containing the same dose as administered on day 0, twice daily starting on day 1, and to recline for 20 minutes after each insertion. On days 14 and 30, blood samples for comparison with the baseline were drawn in the morning while the patients were in a fasting state.

Of the 50 women who participated in the evaluation, 20 were allocated tablets containing 50 mg progesterone, and the remainder of the participants received tablets containing 100 mg progesterone. The baseline details of the participants are summarized in Table I.

TABLE I

| | | Tablets containing | |
|---|---|---|---|
| | Tablets containing | 100 mg | |
| | 50 mg progesterone | progesterone | Total |
| Median age | 43 ± 6.1 | 43.2 ± 7.9 | 43.3 ± 7.2 |
| (years) | | | |
| Age range | 28-53 | 28-5 | 28-55 |
| (years) | | | |
| Height (cm) | 161.3 ± 8.6 | 161.6 ± 5.7 | 161.5 ± 6.9 |
| Weight (kg) | 67.1 ± 11. | 62.8 ± 13.1 | 64.5 ± 12.5 |
| BMI (kg/m²) | 25.9 ± 4. | 24.0 ± 4.4 | 24.8 ± 4.4 |

Data are expressed as mean ± standard deviation unless otherwise specified. Body mass index (BMI) was calculated as weight in kg divided by the square of height in meters.

A single vaginal application of a 50 mg progesterone-containing tablet prepared in accordance with the invention resulted in the rapid increase of plasma progesterone concentration. The mean peak plasma level (T_{MAX}), mean elimination half-life (T_{1/2}), maximal serum concentration (C_{MAX}), and AUC (area under the curve, i.e. total amount of plasma progesterone observed) derived from the blood samples taken on day 0 of the evaluation are summarized in Table II.

TABLE II

| | Progesterone dose | |
|---|---|---|
| | 50 mg (20 patients) | 100 mg (30 patients) |
| Tₘₐₓ (hours) | 6.1 ± 2.63 | 6.4 ± 3.35 |
| T_{1/2} hours | 13.18 ± 1.3 | 13.7 ± 1.05 |
| Cₘₐₓ (nmol/liter) | 20.43 ± 8.01 | 31.61 ± 12.62^{a} |
| AUC (nmol/hour/liter) | 154.15 ± 60.31 | 247.61 ± 123.04^{b} |

Values are mean ± standard deviation;
^{a}P = 0.0004;
^{b}P = 0.001.

As shown in Table III, after 14 and 30 days of continuous application twice daily, the serum P levels were significantly higher compared to baseline values on day 0. No statistically significant difference in plasma levels of follicle stimulating hormone, leutinizing hormone, estradiol, cortisol, dehydroepiendosterone sulfate, or aldosterone were observed in the study groups between baseline values and after continued administration of the tablets of the invention. Similarly, the plasma levels of serum glutamic oxaloacetic transaminase, alkaline phosphatase, cholesterol, triglycerides, high density lipoprotein, low density lipoprotein, and very low density lipoprotein did not change significantly between the baseline measurement and the measurements at 14 and 30 days of twice-daily administration.

TABLE III
Blood Progesterone levels, nmol/liter

| | Progesterone dose | |
|---|---|---|
| Day sample was taken | 50 mg (20 patients) | 100 mg (30 patients) |
| Day 0^{a} | 1.05 ± 0.7 | 3.0 ± 2.4 |
| Day 14^{a} | 17.48 ± 9.8^{b} | 26.08 ± 13.96^{b} |
| Day 30^{a} | 17.38 ± 14.39 | 21.42 ± 16.32 |

^{a}P = 0.0001, significant difference between progesterone baseline values on day 0 compared to day 14 and day 30;
^{b}P = 0.02.

### EXAMPLE 5

The efficacy of tablets prepared in accordance with the present invention was compared with the efficacy of prior art tablets as follows:

Thirteen healthy, postmenopausal women with intact uteri who were undergoing hormonal replacement therapy (HRT) were given complete medical evaluation by history, physical and gynecological examination, and instructed to discontinue HRT two weeks prior to the comparative trial.

Part A: Single-dose pharmacokinetics of micronized progesterone in the form of a gelatin capsule (Utrogestan, produced by Basins-Iscovesco, Paris, France). Participants received oral ethinyl estradiol (Estrofem, Novo-Nordisk, Denmark), 4 mg per day for 14 days. On day 14 at 8 AM, in a fasting state, an intravenous indwelling catheter was inserted into the cubital vein and blood was drawn for baseline progesterone and estrogen levels. The women were then instructed to self-administer a single gelatin capsule containing 100 mg of micronized progesterone high in the vagina. Repeat blood samples for progesterone concentrations were drawn ½, 1, 2, 4, 6, 8, 10, 12 and 24 hours after the vaginal insertion.

Part B: Single-dose pharmacokinetics of micronized progesterone in the form of a vaginal tablet according to the present invention. After a washout period of 2 weeks, the same patients as in Part A were again administered 4 mg or ethinyl estradiol (Estrofern) for 14 days. On day 14 the same procedure as recited in Part A was repeated, except that this time the women were instructed to insert 100 mg of progesterone in the form of an effervescent tablet according to the present invention, using a plastic applicator. Blood samples for progesterone levels were drawn at the same intervals as in Part A.

Samples were assayed using an Immulite enzyme immunoassay (Diagnostic Products Corporation, Los Angeles, Calif.) to measure plasma progesterone (SI conversion factor 3.18; sensitivity 0.2 ng/ml (0.6 nmol/l, inter- and intra-assay coefficients of variation precision <10%)) and estradiol (E2) (SI conversion factor 3.67; sensitivity 12 pg/ml (44 pmol/l, inter- and intra-assay coefficients of variation precision <10%). The pharmacokinetic parameters calculated from the concentration curve were compared between the two study groups by the Wilcoxon 2-sample test, the Kruskal-Wallis test and by analysis of variance (ANOVA). Students T-test was used to compare estrogen levels for the two treatment parts.

Table IV summarizes the baseline details of the of the thirteen women who participated in the study of Example 5.

TABLE IV

| | Meant ± SD | Median | Minimum | Maximum |
|---|---|---|---|---|
| Age (years) | 52.2 ± 3.6 | 53 | 42 | 57 |
| Weight (kg) | 72 ± 15.4 | 70 | 46 | 100 |
| Height (cm) | 165.1 ± 6.5 | 165 | 155 | 178 |
| BMI (Kg/m²) | 263. ± 4.7 | 25.7 | 19.1 | 34.2 |

Data are expressed as mean ± standard deviation unless otherwise specified. Body mass index (BMI) was calculated as weight in kg divided by the square of height in meters.

The mean peak plasma level (Tₘₐₓ) mean elimination half-life (T_{1/2}), maximal serum concentration (Cₘₐₓ), and AUC (area under the curve, i.e. total amount of plasma progesterone observed) derived from the blood samples taken on day 0 of the evaluation are summarized in Table V.

TABLE V

Treatment

| | Vaginal Tablet | Gelatin Capsule |
|---|---|---|
| Tₘₐₓ (hours) | 6.92 ± 3.12 | 6.23 ± 6.56^{b} |
| T_{1/2} hours | 16.39 ± 5.25 | 22.08 ± 16.5 |
| Cₘₐₓ (nmol/l) | 31.53 ± 9.15 | 23.85 ± 9.57^{a} |
| AUC (nmol/h/l) | 379.99: ± 137.07 | 325.89 ± 167.78 |

Values are mean ± standard deviation,
^{a}P = 0.0472;
^{b}Statistically significant difference of variance, P = 0.02.

A single dose of 100 mg micronized progesterone in the form of both gelatin capsules and vaginally administrable tablets in accordance with the present invention resulted in a similar rapid increase in plasma progesterone levels within 2.5-3 hours after administration. The statistically significant difference of variance between the two groups indicates a more predictable Tₘₐₓ for the tablets of the present invention than for the prior art gelatin capsules.

### EXAMPLE 6

The pharmacokinetics of a vaginal tablet containing micronized progesterone were evaluated in a single-center, randomized, open-label, pharmacokinetic study as follows:

Clinical Methods

Pre-menopausal female patients between 18 and 40 years of age with an intact uterus were randomly assigned to receive vaginal progesterone tablets prepared as in Example 1, in 1 of 2 different dosing regimens (either 100 mg BID, or, according to the present invention, 100 mg TID), or the currently approved treatment, Crinone 8% gel (90 mg QD). Eighteen patients were enrolled (6 per treatment group). The study was divided into 4 phases: Screening, Single-day (single day of dosing), Washout, and Multiple-day (5 days of dosing). During the screening phase volunteers were evaluated for their appropriateness to enroll in the study. Those volunteers who were enrolled were randomized to the treatment groups and initially received one day of treatment with medication (one, two, or three doses over 24 hours depending on their treatment assignment). The washout period, during which time they received no study medication, lasted for seven days. Following the washout, they returned to the clinical for five full days of medication dosing. The approximate duration of patient participation was 13 days, including an overnight visit during the Single-day Phase and a 6-night visit during the Multiple-day Phase. Blood samples for pharmacokinetic analyses were collected over a 48-hour period.

Safety assessments were obtained for all patients enrolled in the study.

Clinical Results

Progesterone serum concentrations increased rapidly following the administration of the vaginal progesterone tablet. Treatment with vaginal progesterone tablets according to this invention produced higher peak concentrations in a shorter time than did Crinone. Surprisingly, the vaginal progesterone tablet TID treatment group attained a higher peak concentration than the vaginal progesterone tablet BID treatment group. On the single day of dosing, mean Cmax was 6.82 ng/ml in the Crinone group, and 17 ng/ml in the vaginal progesterone tablet BID group, while the vaginal progesterone tablet TID group reached 19.8 ng/ml. The lowest concentration of the day averaged 40-50% of the peak concentration for all three regimens. Still, the vaginal progesterone tablet regimens according to the methods of this invention provided serum concentrations of progesterone that exceeded the critical 10 ng/ml across the entire Day 5. A mid-luteal phase serum progesterone level of 10 ng/ml (31.8 nmol/l) is associated with adequate corpus luteum function and results in endometrium that is capable of maintaining a pregnancy. Therefore, even between doses and at times when the circulating concentration was at its nadir, levels were maintained which would mimic a healthy natural ovulation cycle. In addition to producing higher serum concentrations (Cmax), both vaginal progesterone tablet regimens produced higher trough levels (Cₘₐₓ) of progesterone, with TID levels superior to BID (C_{trough} on Day 2 of multiple day dosing was 8.82 ng/ml in the BID, 14.5 ng/ml in the TID, and 3.35 ng/ml in the Crinone groups). Similarly, systemic exposure (AUC₀₋₂₄) was greater for the vaginal progesterone tablet according to the present invention, especially for the TID regimen (AUC₀₋₂₄) was 436 ng·hr/ml in the TID, compared to 327 ng·hr/ml for the BID regimen and 264 ng·hr/ml for the Crinone regimen). Among the 3 treatment regimens, vaginal progesterone tablet TID showed the least between-patient variability in Cmax and AUC_{0-τ} whereas Crinone QD showed the greatest between-patient variability.

Vaginal progesterone tablet treatments reached steady-state concentrations more rapidly than did Crinone. Steady-state values were attained within approximately 1 day after initiation of treatment with the vaginal progesterone tablet according to this invention. The progesterone concentrations produced with the vaginal progesterone tablet according to this invention approximated the steady-state progesterone concentrations by the time the second dose was administered in the BID regimen, 12 hours after the first dose, and by the time the second dose on Day 2 was administered in the TID regimen (32 hours after the start of dosing). The TID regimen reaches a higher steady state concentration. Relatively uniform 24-hour concentration time profiles were observed. Based on the steady state data, the effective half-life for approach to steady-state for the vaginal progesterone tablet formulation according to this invention was 4 to 10 hours. The Crinone vaginal gel was estimated to require 6 or more days to reach the steady-state serum concentrations, implying a half-life of 2 days.

Conclusion

The vaginal progesterone tablet regimens produced higher serum concentrations (Cₘₐₓ), higher trough level (C_{trough}), and greater systemic exposure (AUC₀₋₂₄) than did Crinone 90 mg QD. By Day 5 of treatment, both vaginal progesterone tablet regimens provided serum concentrations of progesterone exceeding 10 ng/ml, the level associated with adequate corpus luteum function and which results in endometrium that is capable of maintaining a pregnancy, across the entire Day 5. Surprisingly, the vaginal progesterone tablet TID regimen outperformed both the BID regimen and Crinone regimen in this study. The vaginal progesterone tablet TID treatment attained higher serum concentration Cₘₐₓ and showed the least between-patient variability in Cₘₐₓ and AUC_{0-τ}. Crinone QD showed the greatest between-patient variability.

Progesterone serum concentrations increased rapidly following the administration of the vaginal progesterone tablet vaginal tablet, reaching considerably higher maximum concentration and steady-state values much faster than the Crimone treatment. Though BID and TID treatments produced progesterone serum concentrations that exceeded 10 ng/ml for the entire day, the TID treatment group reached higher maximum concentrations than did the BID group. Thus, the vaginal progesterone tablet provided systemic levels of progesterone consistent with luteal support more quickly and, therefore, for a greater proportion of the therapeutic window. In addition, since progesterone in the tablet form according to this invention is cleared more quickly from the body than Crinone, treatment with the vaginal progesterone formulation according to this invention maintains a more precise therapeutic window, window and can be quickly cleared from the systemic circulation if necessary. Vaginal progesterone tablet vaginal tablets were safe and well tolerated in this healthy patient population.

### EXAMPLE 7

A vaginal micronized progesterone tablet, prepared in accordance with the methods disclosed herein, was compared to Crinone 8% vaginal gel in a multi-center, randomized, open-label, parallel group study. In this study, the test patients were female patients undergoing in-vitro fertilization (IVF). The objective of this study was to determine the efficacy of using a vaginally-administered progesterone tablet to increase pregnancy rates in women undergoing IVF. Safety and tolerability were also examined.

Clinical Methods:

Female patients between 19 and 42 years of age undergoing IVF participated in the study. A total of 1211 patients (the ITT population) were randomized to the three treatment groups: 404 patients received 100 mg twice daily (BID) of a vaginal progesterone tablet according to this invention; 404 patients received 100 mg three times daily (TID) of a vaginal progesterone tablet according to this invention; and 403 patients received Crinone 8% gel (90 mg) once daily (QD). Of these, 36 patients in the ITT population did not undergo embryo transfer and were excluded from the study, leaving an Efficacy population of 1175 patients (392, 390, and 393 patients in the BID, TID and Crinone groups, respectively).

Efficacy Conclusions

Table VI shows the results in the Efficacy Population and Table VII shows the results in the ITT population. In both populations, the group receiving TID treatment with the vaginal progesterone tablet according to this invention achieved higher rates of ongoing pregnancy, biochemical pregnancy and clinical pregnancy than in the BID or Crinone treatment groups.

TABLE VI
Ongoing, Biochemical, and Clinical Pregnancy
Rates - Efficacy Population

| | Vaginal | Vaginal | |
|---|---|---|---|
| | Progesterone | Progesterone | Crinone |
| | Tablet | Tablet | 8% gel |
| | 100 mg BID | 100 mg TID | 90 mg QD |
| Pregnancy Rates | (N = 392) | (N = 390) | (N = 393) |
| Ongoing | 156 (40%) | 171 (44%) | 170 (43%) |
| Pregnancy Rate | | | |
| Biochemical | 198 (51%) | 225 (58%) | 212 (54%) |
| Pregnancy Rate | | | |
| Clinical | 163 (42%) | 183 (47%) | 174 (44%) |
| Pregnancy Rate | | | |

TABLE VII
Ongoing, Biochemical, and Clinical
Pregnancy Rates - ITT Population

| | Vaginal | Vaginal | |
|---|---|---|---|
| | Progesterone | Progesterone | Crinone |
| | Tablet | Tablet | 8% gel |
| | 100 mg BID | 100 mg TID | 90 mg QD |
| Pregnancy Rates | (N = 404) | (N = 404) | (N = 403) |
| Ongoing Pregnancy Rate | 156 (39%) | 171 (42%) | 170 (42%) |
| Biochemical Pregnancy Rate | 198 (49%) | 225 (56%) | 212 (53%) |
| Clinical Pregnancy Rate | 163 (40%) | 183 (45%) | 174 (43%) |

### EXAMPLE 8

Many factors affect fertility and impact the success rates of infertility treatments. Notably, age and ovarian reserve (as reflected by serum FSH) have strong correlations with pregnancy success rates and are primary clinical factors in determining treatment regimens that are tailored for each patient. For example, the 2003 Society for Assisted Reproductive Technology (SART) database indicates that the birth rate following IVF for women younger than 35 years was 37%, but was only 11% for women 41 to 42 years of age.

In this Example, the efficacy of a vaginal progesterone tablet made according to Example 1, and of Crinone, was studied in the female patients from Example 7 who were between 35 and 42 years of age, as follows: 157 patients received 100 mg twice daily (BID) of a vaginal progesterone tablet according to this invention; 157 patients received 100 mg three times daily (TID) of a vaginal progesterone tablet according to this invention; and 160 patients received Crinone 8% gel (90 mg) once daily (QD).

Efficacy Conclusions

Table VIII contains the results from this study. Surprisingly, TID treatment with the vaginal progesterone tablet according to the present invention resulted in much higher rates of ongoing pregnancy, biochemical pregnancy and clinical pregnancy than in the BID group, for patients 35-42 years old.

TABLE VIII
Ongoing, Biochemical, and Clinical Pregnancy
Rates in Patients 35-42 Years of Age

| | Vaginal | Vaginal | |
|---|---|---|---|
| | Progesterone | Progesterone | Crinone |
| | Tablet | Tablet | 8% gel |
| Pregnancy Rates | 100 mg BID | 100 mg TID | 90 mg QD |
| Patients 35-42 Years of Age | (N = 157) | (N = 157) | (N = 160) |
| Ongoing Pregnancy Rate | 45 (29%) | 54 (34%) | 62 (39%) |
| Biochemical Pregnancy Rate | 56 (36%) | 79 (50%) | 76 (48%) |
| Clinical Pregnancy Rate | 46 (29%) | 62 (39%) | 65 (41%) |

Table VIII establishes that TID treatment of older female patients with a vaginal progesterone tablet according to this invention is far superior to BID treatment with the same vaginal progesterone tablet in older female patients. It is possible that the TID regime exerts a greater effect in older patients and in patients with diminished ovarian reserve, who require stronger luteal support.

### EXAMPLE 9

The efficacy of a vaginal progesterone tablet made according to Example 1, and of Crinone, was studied in the female patients from Example 7 who were overweight (i.e., having a BMI between about 25.0 and about 29.9 kg/m²) or obese (i.e., having a BMI≧about 30 kg/m²). For the “Overweight group,” 121 patients received 100 mg twice daily (BID) of a vaginal progesterone tablet according to this invention; 120 patients received 100 mg three times daily (TID) of a vaginal progesterone tablet according to this invention; and 112 patients received Crinone 8% gel (90 mg) once daily (QD). For the “Obese group”: 66 patients received 100 mg twice daily (BID) of a vaginal progesterone tablet according to this invention; 75 patients received 100 mg three times daily (TID) of a vaginal progesterone tablet according to this invention; and 66 patients received Crinone 8% gel (90 mg) once daily (QD).

Table IX, below, shows the results of this study. Biochemical Pregnancy Rate (i.e., pregnancy as indicated by a positive result in a serum pregnancy test) was determined in the both test groups.

TABLE IX
Biochemical Pregnancy Rate by Body Mass Index - ITT Population

| | | | Vaginal | Vaginal | |
|---|---|---|---|---|---|
| | | | progesterone | progesterone | Crinone |
| | Body Mass Index | | tablet | tablet | 8% gel |
| | (BMI) | | 100 mg BID | 100 mg TID | 90 mg QD |
| | Categories | | (N = 404) | (N = 404) | (N = 403) |
| Biochemical Pregnancy Rate | 25.0-29.9 | kg/m² | (n = 121) | (n = 120) | (n = 112) |
| | | | 55 (45%) | 69 (58%) | 54 (48%) |
| Biochemical Pregnancy Rate | ≧30 | kg/m² | (n = 66) | (n = 75) | (n = 66) |
| | | | 29 (44%) | 37 (49%) | 33 (50%) |

The results in Table IX tends to establish that TID treatment of overweight or obese female patients with a vaginal progesterone tablet according to this invention is superior to Crinone treatment or BID treatment with the same vaginal progesterone tablet in overweight or obese/morbidly obese females.

### EXAMPLE 10

It is expected that women older than 42 years old we might require even stronger luteal support than women between the ages of 35 and 42 years old. Therefore, in addition to the use of a vaginal progesterone tablet made according to the present invention for the treatment of women between the ages of 35 and 42, as described in Example 8, above, it is expected that the advantages of TID treatment with a vaginal progesterone tablet made according to the methods disclosed herein would be more enhanced in women older than 42 years old.

Accordingly, the efficacy of a vaginal progesterone tablet made according to the present invention could be studied in the female patients over 42 years of age, using the same, or similar, test parameters as those used in the studies described in Examples 7, 8 and 9.

Pregnancy rates are shown to be higher when Endometrin is administered TID rather than BID. This is more pronounced in women 35-42 years old than in women under age 35. It is thus expected that women older than 42 years of age may require more luteal support than women younger than 42. Therefore, it is anticipated that TID treatment of women older than 42 years old with a vaginal progesterone tablet made according to the methods disclosed herein will result in higher rates of ongoing pregnancy, biochemical pregnancy and clinical pregnancy than in women older than 42 years of age who are treated BID with the same vaginal progesterone tablet.

### EXAMPLE 11

Older women and women who have poor ovarian reserve are often presented with the option of using donor oocyte in order to achieve pregnancy. Such women are often older, i.e., older than 42 years of age. Some may also be overweight (i.e., having a BMI between about 25.0 and about 29.9 kg/m²), or obese (i.e., having a BMI≧about 30 kg/m²). These women have already shown themselves to have poor ovarian function. In addition, by using a donor oocyte, these women have not stimulated their ovaries to maximize the production of endogenous hormones. Therefore, women who enter a donor oocyte program will likely require stronger luteal support than women undergoing standard IVF. Accordingly, in addition to the use of a vaginal progesterone tablet made according to the methods disclosed herein in women being treated for infertility or other pregnancy-related conditions and disorders in an IVF program, as described in Examples 7, 8 and 9, above, it is anticipated that the advantages of TID treatment with a vaginal progesterone tablet made according to the methods disclosed herein would be more enhanced in women in a donor oocyte program.

Accordingly, the efficacy of a TID treatment program with a vaginal progesterone tablet made according to the methods disclosed herein could be studied in the female patients undergoing donor oocyte to achieve pregnancy.

Thus, woman in donor oocyte programs will likely require more luteal support than women in IVF programs. Additionally, women undergoing donor oocyte IVF are often older (e.g., more than 35 years of age), and it is apparent from Example 8 that older women benefit from TID administration of a vaginal progesterone tablet. Therefore, it is anticipated that TID treatment with a vaginal progesterone tablet made according to the methods disclosed herein in women undergoing donor oocyte treatment will result in higher rates of ongoing pregnancy, biochemical pregnancy and clinical pregnancy than in women undergoing donor oocyte treatment who are treated BID with the same vaginal progesterone tablet.

It is to be understood that the amounts and proportions of ingredients recited in the foregoing examples are illustrative only, and that these amounts and proportions may be varied within the scope of the invention. For example, the Example 1 the amount of effervescent recited is about 8 wt. % of the tablets which are the final product of the process described in Example 1. However, the effervescent may be omitted in the practice of the invention, or it may be included in an amount of up to about 12 wt. % of the tablet. Preferably the effervescent constitutes between about 5-12 wt. %, more preferably between about 6-8 wt. % of the tablet. Similarly, progesterone may constitute up to about 20 wt. % of the tablet, preferably between about 6-20 wt. %, more preferably between about 8-12 wt. % of the tablet.

It will be appreciated that various features of the invention which are, for clarity, described in the contexts of separate embodiments may also be provided in combination in a single embodiment. Conversely, various features of the invention which are, for brevity, described in the context of a single embodiment may also be provided separately or in any suitable subcombination.

It will also be appreciated by persons skilled in the art that the present invention is not limited by what has been particularly shown and described hereinabove. Rather the scope of the invention is defined only by the claims which follow:

What is claimed is:

1. A method of delivering progesterone to a female patient for hormone replacement therapy, which comprises placing in the vagina of said patient at least three times daily a tablet comprising progesterone as the active ingredient, pharmaceutically acceptable excipients or diluents, and an effervescent,
and retaining said tablet in the vagina for a time efficacious to deliver said progesterone to said patient.

2. A method according to claim 1, wherein the progesterone in said tablet is present in an amount of at least about 50 mg.

3. The method according to claim 1, wherein the progesterone in said tablet is present in an amount of at least about 100 mg.

4. The method of claim 1, wherein the progesterone in said tablet is micronized.

5. The method of claim 1, wherein the effervescent in the tablet is present in an amount of about 5% to about 12% by weight of the tablet.

6. The method of claim 1, wherein the effervescent in the tablet is present in an amount of about 8% by weight of the tablet.

7. A method of delivering progesterone to a female patient, which comprises placing in the vagina of said patient at least three times daily a tablet comprising progesterone, a pharmaceutically acceptable excipient or diluent, and an effervescent, wherein said tablet is prepared by the steps of:
(i) mixing water with progesterone to obtain wetted progesterone in the absence of pharmaceutically acceptable excipients or diluents; and drying said wetted progesterone to form dry progesterone;
(ii) mixing said dry progesterone with
(a) a pharmaceutically acceptable excipients or diluent and
(b) an effervescent to form a mixture; and
(iii) forming the tablet by direct compaction of said mixture, and retaining said tablet in said vagina until the tablet dissolves, wherein the tablet provides a Tₘₐₓ upon dissolution of about six hours.

8. A method according to claim 7, wherein the progesterone in said tablet is present in an amount of at least about 50 mg.

9. A method according to claim 7, wherein the progesterone in said tablet is present in an amount of at least about 100 mg.

10. The method of claim 7, wherein the progesterone in said tablet is micronized.

11. The method of claim 7, wherein the effervescent in the tablet is present in an amount of about 5% to about 12% by weight of the tablet.

12. The method of claim 7, wherein the effervescent in the tablet is present in an amount of about 8% by weight of the tablet.

13. A method of delivering progesterone to a female patient, which comprises
(a) placing in the vagina of the patient at least three times daily a vaginal tablet comprising micronized progesterone as the active ingredient, pharmaceutically acceptable excipients or diluents, and an effervescent; and
(b) permitting the tablet to dissolve in the vagina, the tablet providing a Tₘₐₓ of about six hours upon dissolution.

14. A method according to claim 13, wherein the progesterone in said tablet is present in an amount of at least about 50 mg.

15. A method according to claim 13, wherein the progesterone in said tablet is present in an amount of at least about 100 mg.

16. The method of claim 13, wherein the progesterone in said tablet is micronized.

17. The method of claim 13, wherein the effervescent in the tablet is present in an amount of about 5% to about 12% by weight of the tablet.

18. The method of claim 13, wherein the effervescent in the tablet is present in an amount of about 8% by weight of the tablet.

19. A method of delivering progesterone to a female patient, comprising placing in the vagina of said patient at least three times daily a tablet consisting essentially of progesterone as the active ingredient, pharmaceutically acceptable excipients or diluents, and an effervescent, and retaining said tablet in the vagina for a time efficacious to deliver said progesterone to said patient.

20. The method according to claim 1, wherein said female patient is ≧ about 35 years old.

21. The method according to claim 20, wherein said female patient is between about 35 and about 42 years old.

22. The method according to claim 1, wherein said female patient has a Body Mass Index (BMI) between about 25 and about 29 kg/m².

23. The method according to claim 1, wherein said female patient has a Body Mass Index (BMI) that is ≧ about 30 kg/m².

24. The method according to claim 1, wherein said female patient is ≧ about 42 years old.

25. The method according to claim 1, wherein said female patient is undergoing donor oocyte treatment.
